(19) United States
(12) Patent Application Publication
(10) Pub. No.: US 2026/0199051 A1
(43) Pub. Date: Jul. 16, 2026

(54) OPTICALLY RESPONSIVE MEDICAL-GRADE MATERIALS, ARTICLES, SYSTEMS, AND METHODS FOR MACHINE-DETECTABLE CONDITION INDICATION

(71) Applicant: Mary SHERWOOD, Rancho Sante Fe, CA (US)

(72) Inventor: Mary SHERWOOD, Rancho Sante Fe, CA (US)

(21) Appl. No.: 19/447,263

(22) Filed: Jan. 13, 2026

Related U.S. Application Data
(60) Provisional application No. 63/744,497, filed on Jan. 13, 2025.
(60) Provisional application No. 63/958,708, filed on Jan. 12, 2026.

(51) Int. Cl. A61B 90/00

(52) U.S. Cl. CPC A61B 90/39; A61B 2090/3945

(57) ABSTRACT

An optically responsive material/device and methods for its manufacture and application are disclosed. The material comprises an optically responsive component, which may include luminescent particles, incorporated or encapsulated within a supporting structure and configured to exhibit a detectable optical characteristic, including light emission in low-visibility environments. The material is suitable for use in medical tubing, catheters, surgical instruments, nasal cannulas, wound dressings, medical implants, and other medical and non-medical devices, and improves visibility, safety, and usability during medical procedures. In various embodiments, the material provides indication of operational conditions while maintaining biocompatibility and mechanical integrity. Additional features may include antimicrobial properties, customizable optical intensity, variable emission spectra, and color variations. The material may further be configured to integrate with external scanning or detection devices capable of reading embedded or associated data, enabling monitoring, identification, and procedural tracking.

### CROSS-REFERENCE TO RELATED APPLICATION(S)

This application claims the benefit and priority of U.S. Provisional patent application Ser. No. 63/744,497, titled “Luminous Medical-Grade Material for Use in Medical Tubing, Catheters, Surgical Instruments, and Medical Devices,” filed Jan. 13, 2025, and U.S. Provisional Patent Application Ser. No. 63/958,708 , titled “Optically Responsive Materials and Devices for Machine-Detectable Condition Indication” filed Jan. 12, 2026 the contents of which are hereby incorporated by reference in their entirety.

### FIELD

The invention relates to an optically responsive device and method for visual and status indication for industrial, commercial, and medical environments. In particular, the optically responsive device and method are well adapted for medical tubing, catheters, surgical instruments, nasal cannulas, wound dressings, implants, and other medical devices, including larger systems and methods implementing the same, wherein optical responsiveness may include luminous behavior.

### BACKGROUND

Modern medical procedures require precision and safety, often performed in dimly lit or low-visibility environments. Standard medical-grade materials lack optically responsive and/or self-luminescent properties, making it challenging to identify or use medical devices effectively, including assessing operational conditions such as flow. This invention addresses this gap by providing an optically responsive medical-grade material, which may exhibit luminous behavior, to enhance visibility and provide visual indication of operational conditions while maintaining biocompatibility, mechanical strength, and functionality.

Medical professionals often encounter difficulties in quickly locating medical devices, catheters, or tubing, particularly during emergencies or in darkened surgical fields. This limitation can lead to delays, errors, and increased risks for patients. Existing solutions rely on external light sources, reflective coatings, or manual tagging, which have inherent limitations such as battery dependence, external light requirements, or degradation over time. The present invention eliminates these drawbacks by offering a self-luminous configuration that provides sustained visibility without requiring external power sources. Additionally, by incorporating embedded readable data, the material allows for seamless integration with external scanning devices, ensuring efficient medical tracking and enhanced safety.

### SUMMARY

The following presents a simplified summary in order to provide a basic understanding of some aspects of the claimed subject matter. This summary is not an extensive overview and is not intended to identify key/critical elements or to delineate the scope of the claimed subject matter. Its purpose is to present some concepts in a simplified form as a prelude to the more detailed description that is presented later.

In various embodiments, an optically responsive medical-grade material for manufacturing medical tubing, catheters, surgical instruments, nasal cannulas, wound dressings, implants, and other medical and non-medical devices is described, wherein the optical responsiveness may include luminous behavior. The material can be comprised, in some non-limiting examples, of a biocompatible base polymer infused with optically responsive particles. In other examples, it can be a coating or infusion of optically responsive material in or on a surface of the device.

In various embodiments, the material can be activated by exposure to ambient or ultraviolet light and retains its luminescence for extended periods. In other embodiments, unique identifiers and data storage elements are implemented, allowing for real-time scanning and external device communication for tracking and procedural monitoring. Aspects of providing enhanced visibility, antimicrobial properties, customizable luminescence, multi-color emission, and prolonged emission duration, and so forth are detailed below, ensuring its utility across diverse medical and non-medical applications.

In one aspect of the disclosed embodiments, an optically responsive medical-grade article is provided, comprising: a body formed of a polymeric material suitable for use in a medical environment; and an optically responsive component embedded within the body, wherein the optically responsive component is configured to produce a machine-detectable optical response in response to an operational condition of the article, and wherein the optical response is configured to be detected by a sensing system and used to associate information indicative of the operational condition, without reliance on human visual observation.

In another aspect of the disclosed embodiments, the above optically responsive medical-grade article is provided, wherein the optically responsive component comprises luminescent particles dispersed within the body; and/or the luminescent particles comprise phosphorescent, fluorescent, electroluminescent, or triboluminescent materials; and/or wherein the optically responsive component is embedded in a manner that inhibits leaching, migration, or degradation during use; and/or the machine-detectable optical response varies in response to at least one operational condition selected from fluid flow, pressure, temperature, movement, or chemical exposure; and/or wherein the optically responsive component is arranged in a defined region configured to encode information detectable by the sensing system; and/or wherein the optically responsive component further exhibits an electrostatic or capacitive characteristic detectable by the sensing system; and/or wherein the optically responsive component further exhibits a magnetic characteristic detectable independently of visible light emission; and/or wherein the article comprises medical tubing, a catheter, a cannula, or a conduit; and/or wherein the conduit defines a lumen configured to convey a fluid.

In yet another aspect of the disclosed embodiments, a system is provided, comprising: the optically responsive medical-grade article of above; and a sensing system configured to detect the machine-detectable optical response of the article produced by the article and to associate the detected optical response with information indicative of the operational condition of the article; and/or wherein the sensing system comprises an optical sensor, a camera, or a machine-vision device; and/or further comprising a processor configured to process the associated information; and/or wherein the processor generates a signal based on the associated information.

In another aspect of the disclosed embodiments, a method of monitoring an operational condition of a medical-grade article is provided, comprising: providing the optically responsive medical-grade article of above; subjecting the article to an operational condition; detecting, using a sensing system, a machine-detectable optical response produced by the article; and associating the detected response with information indicative of the operational condition; and/or further comprising generating a signal based on the associated information; and/or comprising using the signal to control or adjust operation of a medical device or delivery system; and/or wherein the detection is performed without reliance on human visual observation; and/or the associated information is used for workflow verification, delivery confirmation, or device status validation; and/or wherein the associated information is used to trigger an alert, record a verification event, or modify an automated process; and/or wherein detecting is performed without reliance on human visual observation; and/or further comprising processing the associated information to generate a signal; and/or further comprising using the signal to verify or influence operation of a medical device or process associated with the article; and/or wherein detection occurs continuously, periodically, intermittently, or in response to a triggering condition; and/or wherein the associated information is recorded or stored as part of a verification or documentation process.

Additional and alternative descriptions are provided below.

### BRIEF DESCRIPTION OF THE DRAWINGS

FIG. 1 is a generic illustration of an exemplary embodiment.

FIG. 2 is a generic illustration of an exemplary cover or sleeve embodiment.

FIG. 3 is a block diagram of an exemplary embodiment connected to an attendant device.

FIG. 4 is an illustration of an exemplary embodiment for use as a cannula body for an oxygen delivery tube.

FIG. 5 is an illustration of an exemplary embodiment being used by a patient.

### DETAILED DESCRIPTION

Medical grade is a term of art understood to quantify a product as suitable for medical use. Narrowly speaking, testing standards according to ISO: 10993-5/10 and ISO: 13485 may operate as official standards for applying a medical grade qualification. However, broadly speaking, the term may be applied for a product considered to be safe for use in the administration of medical care (e.g. a Q-tip). In the context of this disclosure, the term medical grade will mean a product or goods/service suitable in any of the above standards, but exampled below as used in a hospital or a like-caring capacity, wherein reasonable health and sanitation standards are followed.

Notwithstanding the above, it is expressly understood that while the below descriptions are presented in the context of a medical use, the devices and methods described are also applicable to other venues, not being limited to the medical arts. For example, industrial, commercial, leisurely and other environments may benefit from the invention.

As alluded above, a device and method for a luminous medical-grade material for use in medical tubing, catheters, surgical instruments, nasal cannulas, wound dressings, implants, and other medical or non-medical devices is described. With one or more of enhanced visibility, antimicrobial properties, customizable luminescence, multi-color emission, and prolonged emission duration, ensuring its utility across diverse applications. The exemplary material can be activated by exposure to ambient or ultraviolet light and retains its luminescence for extended periods. Additionally, the luminous material can be formulated from nanocomposites, polymers, multilayered structures, and controlled emission profiles. And may come in a powder form, or as a gel, polymer, coatings, beads, etc. In one exemplary embodiment, the luminous material is created by integrating luminescent particles into a biocompatible base polymer, for example, encapsulating the luminescent particles. When integrated as encapsulated luminescent particles, leaching can be prevented, ensuring long-term safety and efficacy. Additionally, unique identifiers and data storage elements can be incorporated, allowing for real-time scanning and external device communication for tracking and procedural monitoring.

The luminescent particles (or EM emitting particles) can be selected from phosphorescent, fluorescent, triboluminescent, chemiluminescent, or electro/magno-luminescent materials, ensuring prolonged light emission after exposure to a light source. The material can be further enhanced with antimicrobial agents to prevent bacterial growth, radiation resistance for sterilization compatibility, and structural reinforcement to maintain durability under medical conditions.

In various embodiments, the luminescent particles can layered on top of non-luminescent material (not necessarily embedded therein) and shielded from external contact via a covering or sleeve, typically a transparent (e.g., non-light blocking) or nearly transparent waterproof covering. The covering may be in some instances permanent, according to design preferences. In some embodiments, the covering may act as a light filter. In other embodiments, the covering may act as a light frequency shifter, thus changing a viewed color from the actual color emitted by the luminescent particles. A non-limiting example is the use of quantum dots or a color filter. In other embodiments, the covering may be selective in coverage and/or light filtering/shifting. As a non-limiting example, the covering may have one side that has an altered color viewable while another side is 100% transparent. In another example, the covering may be moveable in some manner, e.g., rotate or slide to reveal or cover the luminescent particles, thus providing a mechanical means for altering a viewed “status” of the luminescent particles (or the medical device having the electroluminescent particles). It is expressly understood that some types of materials can serve as light enhancers/distributors and therefore surrounding material of the luminescent particles may be of such a material to “amplify” light or direct it to a desired area. Fiber optics, Fresnel lens, waveguides and so forth are non-limiting examples of such enhancers/distributors.

In other embodiments, the luminescent particles may be embedded into a “coupler” and the coupler serves as the means to enhance an existing device to have the above/below-mentioned capabilities. Thus, the term “covering” used above may also be applicable (as to implementation) in the form of a coupler-like embodiment. As a non-limiting example, a drip delivery cannula may have its liquid/drip port attached to an exemplary coupler embodiment and the exemplary coupler then connected to a mediation/plasma bag tube line. Conversely, the exemplary coupler may be introduced anywhere else within the delivery system. In another non-limiting example, an exemplary luminescent nasal cannula in an oxygen delivery system can be the piece replaced in the delivery system. Thus, in this example no major replacement of the existing infrastructure is needed to obtain the desired features.

The following list provides various features, some which can be in combination with other features or stand alone, depending on the conditions under use and device type:
Luminescent Composition: Fluorescence can be achieved via any one or more of a phosphorescent, fluorescent, luminescent, radioluminescent, triboluminescent, or electroluminescent particles embedded within the material (or layered on) to provide sustained visibility in low-light conditions. The body of such luminescent particles are too numerous to list (and are changing monthly) but are readily available to one of ordinary skill in the art, and while not explicitly named herein are understood to be within the purview of this disclosure, being incorporated herein by reference. For the purposes of ease of reference, the term luminescent will refer to any one of the above-mentioned light emitting particles, noting in the context of this disclosure, the light emitting particle may be of a passive material. In some embodiments, in addition to natural light, UV or some variant of EM light, an isotopic radiative material may be used as an energy source for providing activation of the luminescent particles. In some embodiments, the luminescent particles may contain magnetic particles with non-visible properties, wherein a magnetic signature is provided by the particles. Additionally, if the luminescent particles are magno-luminescent, then the presence of magnetic particles can operate as an activation factor, according to design and implementation preference. In some embodiments, the luminescent particles may contain particles that are acoustically responsive, thus another form of non-light communication can be facilitated. In either or all of these embodiments, a specific arrangement (e.g., geometry/pattern) of the particles can be made, to provide additional capabilities beyond simply a bulk response.
Biocompatibility: Medical caliber ensures safe interaction with human tissue, bodily fluids, and medical environments.
Customizable Luminescence: Adjustable intensity, color variations, and emission duration based on particle selection and concentration. A non-limiting example is a temperature sensitive luminescent, wherein color or sensitivity to UV light would alter according to the temperature. Another non-limiting example is pressure sensitivity, wherein a color or brightness changes according to an experienced pressure (or tension/stress/force).
Antimicrobial Properties: Integrated antimicrobial agents to reduce bacterial adhesion and infection risk. A non-limiting example would be the use of silver impregnation and so forth.

Encapsulation Technology: Encapsulated luminescent particles prevent leaching or degradation over time. Encapsulation can also take the form of being embedded into a medical device “cover” wherein the cover is slipped (or attached/secured) onto or into an element of the device.
Sterilization Compatibility: Maintains luminescent properties after exposure to radiation, autoclaving, or chemical sterilization.
Multi-Layered Applications: Can be used as coatings, embedded layers, or mixed polymers for enhanced functionality.
Flexible and Rigid Configurations: Adaptable to flexible tubing, rigid surgical instruments, and implantable devices.
Data Integration for External Scanning: The material can be embedded with RFID, NFC, or other data-readable elements that can be scanned externally for real-time medical data retrieval. Patterns of the luminescent particles can be made for scanning or other purposes.
Procedure and Patient Tracking: Due to the optical (or EM/acoustic) signature available, “reading” the device enables seamless monitoring of implanted medical devices, ensuring improved identification, procedural tracking, and post-surgical follow-up. As a non-limiting example, a luminescent bar or scan-like luminescent code may be utilized.

In view of the above features, numerous applications are available. The following is a non-exhaustive list of easy-to-implement uses in the context of medical services:
Medical Tubing: For intravenous lines, respiratory lines, suction systems, and dialysis tubing.
Catheters: Visible catheters for insertion, navigation, and monitoring.
Surgical Instruments: Enhanced precision and quick identification in dim environments.
Nasal Cannulas: Improved identification for emergency use and oxygen delivery.
Wound Dressings: Luminous gauze and bandages for wound care in trauma settings.
Implants: Luminous implantable devices for post-surgical monitoring, patient identification, and procedural tracking.
Endoscopic Tools: Improved visibility for minimally invasive procedures.
External Device Compatibility: Integration with medical scanning devices for real-time information retrieval.

In certain embodiments utilizing tubing as a demonstrative paradigm, one or more sensors can be associated with the tubing and configured to detect physical conditions including, but not limited to:
airflow or gas movement;
respiration-associated temperature changes;
pressure, flow rate, or volume;
interruption, degradation, or loss of flow; and
blockage, occlusion, or dislodgement.

Sensors may be embedded within, layered on, integrated into, or attached to the tubular body, and may be positioned to detect conditions occurring locally along the tubing. Similarly, sensors may be incorporated in a “cover” of the tubular body, if so desired.

In some embodiments, the optical (or EM/acoustic) feature can be used to determine a Condition-Responsive Behavior. For example, a tubing may include one or more optically active regions configured to change a visual characteristic in response to a detected physical condition. Visual characteristics may include changes in color, intensity, pattern, segmentation, or spatial distribution along the tubing. The optical response may be continuous, intermittent, or variable over time. Thus, a direct cause-and-effect relationship at the tubing interface can be visualized, whereby a condition sensed at the tubing causes a corresponding visual change on the tubing itself.

An extension of the above can be applied to zonal and multi-zonal differentiation. That is, different portions of the tubing may exhibit different visual characteristics corresponding to localized conditions occurring along the tubing length. This feature enables simultaneous visual representation of multiple operational states along the tubing and supports detection of partial occlusions, flow variation, or localized dislodgement.

In a medical environment, for example, improper device placement or misalignment can be visually identified using one or more of the features. A luminescent tubing can visually represent proper placement or continued engagement with a patient. For example, temperature or flow signatures associated with respiration may cause visual (color/light) behavior indicating correct placement of tubing or associated portals. As one non-limiting example, luminescent particles may be sensitive to high oxygen presence, and therefore “glow” to show a sufficiency of oxygen, or vice versus, “glow” if there is an insufficiency. Similar examples can be made for carbon dioxide detection and other gases.

Other non-limiting examples of practical medical scenarios are demonstrated: A coupler from an oxygen source to a patient may be improperly connected and glowing may occur for an improper/proper connection, providing a visual cue to the operator or patient. A more specific case can be for oxygen delivery tubing or a nasal cannula. Exemplary sensor(s) can detect airflow or respiration-associated temperature changes, and optically active regions visually indicate oxygen flow or breathing activity directly at the tubing. This implementation enables confirmation of oxygen delivery without reliance on pulse oximetry, capnography, imaging, or external monitors. In certain embodiments, the tubing is configured for suction or drainage. Exemplary sensor(s) detect pressure or flow conditions, and optically active regions visually indicate suction state, blockage, or interruption directly at the tubing. For plasma/medication or drip delivery to a patient, it is not uncommon for the delivery line to be unknowingly constricted from movement by the patient. Thus such a condition can be visually and easily noted. In certain embodiments, an exemplary tubing can provide visual cues during movement, transport, or repositioning of a patient, including use in ambulances, transport carts, emergency settings, or home-care environments. Localized visual feedback remains observable at the tubing despite motion or changes in patient position. Also, in the event of a power failure, the exemplary tubing enables visual representation of an operational state independent of external monitoring equipment and enables an exemplary embodiment to be utilized during low-resource or temporarily disrupted environments.

It is understood many medical devices are single use or multiple use. Similarly, the exemplary embodiments may be manufactured to provide single-use disposable deployment or reusable deployment with sterilization between uses.

In certain embodiments, exemplary embodiment-provided information may be transmitted to or displayed on optional external devices or interfaces for informational, tracking, or documentation purposes. Cameras or EM/acoustic responsive sensors (e.g., to the “light” being emitted/or not emitted) can provide monitoring of the patient's support systems. Thus, reliance on an in-person observation of the embodiment-provided information can be deferred to an automatic system(s).

Accordingly, depending on the complexity of the luminescence material composition (having, in addition to the luminescence particles, other non-luminescence particles) and related sensors, one or more combinations of camera-based sensing, electrostatic sensing, capacitive sensing, electromagnetic sensing, radio-frequency sensing, and acoustic sensing are understood to be feasible.

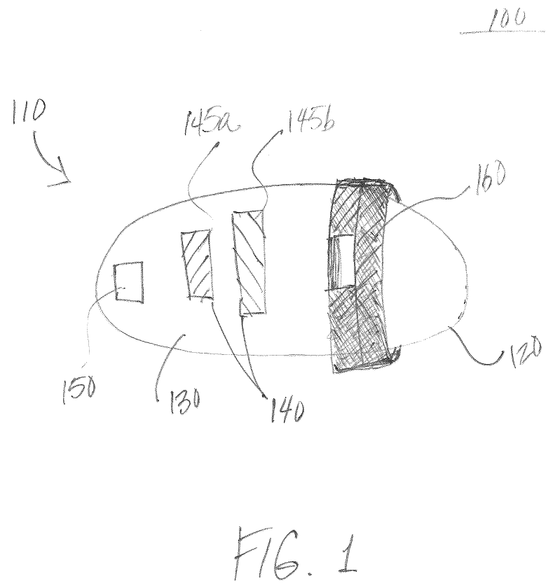

FIG. 1 is a generic illustration 100 of an exemplary embodiment 110. A body 120 is composed of a supporting material 130 having a “luminescent” material (or particles) 140 disposed within or about the supporting material 130. The luminescent material 140 may be of a single type or varied, being concentrated into a contiguous form, gradiented, or separated to form a pattern, shown here are a bands 145, 147. As noted above, the pattern may signify information, such as a scan code or type of information. Different parts of the pattern (e.g., 145a, 145b) may be of a different composition/density of the luminescent material 140 so as to provide different characteristics, as stated previously. Similarly, sensor(s) 150 may be situated in or about the luminescent material 140 to detect an optical condition triggered by the luminescent material 140. These sensor(s) 150 may also serve to alter the light type or light characteristic of the luminescent material 140 or may operate to signal to local devices/systems of a detected state of the luminescent material 140. As a non-limiting example, a sensor(s) 150 may EM-transmit to a nearby station of a triggering event. Accordingly, such a sensor(s) 150 may be optically response, and/or utilize NFC, RFID, optical, Bluetooth and etc. modalities to achieve local communication.

The body 120 may be in a tube form, solid, coiled, varied, hollow and so forth. It is understood, if used for medical purposes, the body 120 can be of a medical grade caliber. The body 120 can be configured with different optical (or EM/acoustic) characteristics at different locations within the body 120. The observed optical characteristic can vary due to the body 120 (composition, geometry, shape, etc.) or the “luminescent” material 140 (composition, geometry, shape, etc.) being distributed within the body 120. Thus, is it contemplated one side of the body 120 may emit more (or different) “light” than an other side.

In addition, FIG. 1 shows an optional cover (or sleeve) 160 which can be “slid” over the “luminescent” material 140 to block or alter an emission state or characteristic of the “luminescent material” 140. As described earlier, the cover 160 may have a varying shape. In some embodiments, the cover 160 may also have “luminescent material” 140 either of a similar type or of a different type, or an activation element therein. In some embodiments, the cover 160 may contain sensor(s) 150, whereas movement of the cover 160 brings the sensor(s) 150 into proximity of the “luminescent” material 140. While FIG. 1 shows a singular exemplary embodiment 110, it is understood that several forms of the exemplary embodiment 110 may be concatenated or distributed within the environment of deployment.

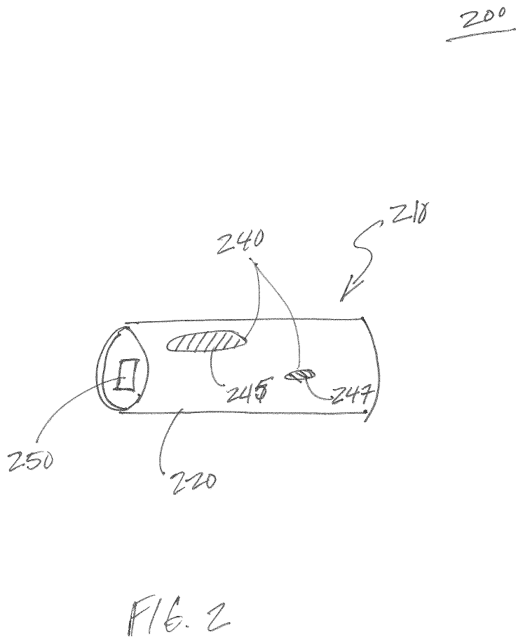

FIG. 2 is a generic illustration 200 of an exemplary cover or sleeve embodiment 210 having one or more of the attributes of the exemplary embodiment 110 of FIG. 1. Cover/sleeve body 220 can having a “luminescent” material 240 disposed within or about the cover body 220. The “luminescent” material 240 may be of a single type or varied, concentrated into a contiguous form, gradiented, or separated to form a pattern 245, 247. Sensor(s) 250 may be situated in or about the “luminescent” material 240. The cover body 220 may be placed over or into (if configured as a coupler) within an existing system. As a non-limiting example, as a “luminescent” sleeve on a scalpel, or as a “luminescent” coupler.

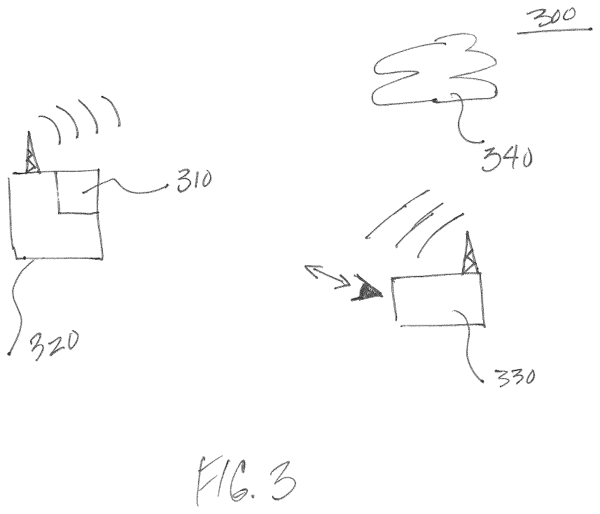

FIG. 3 is a block diagram 300 of an exemplary embodiment 310 connected to an attendant device(s) 320. This example includes an exemplary embodiment monitoring system 330 which can be a camera, UV/energy providing source, or embodiment responsive device, capable of detecting either light from the exemplary embodiment 310 or some other characteristic arising to the nature of the underlying particles within or attached to the exemplary embodiment 310, as discussed above. The attendant device(s) 320 or monitoring system 330 may further be connected to an external communication network 340, wherein such a communication network 340 can signal an attendant or medical professional (not shown), or to an ancillary system, as needed. As stated above, scanning capability can be a function of the monitoring system 330. Wherein information from the scan is forwarded to the external communication network 340, upon which evaluation of the information is acted upon. It is expressly understood aspects of this embodiment include computing capabilities, software, databases, processors, memory, etc. which are either part of, or connected to the external communication network 340 and may similarly be applicable to attendant device(s) 320.

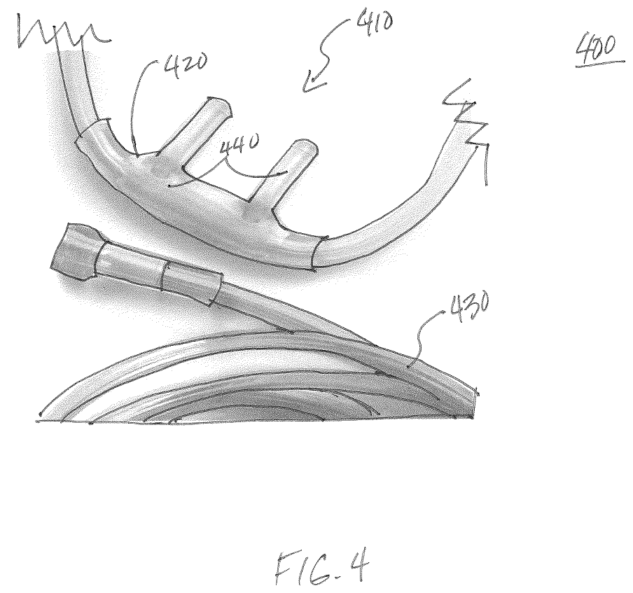

FIG. 4 is an illustration 400 of an exemplary embodiment 410 for medical-directed use as a cannula body 420 with an oxygen delivery tube 430 (shown here in coiled and cuttoff form). The exemplary cannula body 420, in this example has luminescent material 440 (and/or additional particles that are non-luminescent) disposed within an entirety of the cannula body 420. The cannula body 420 and luminescent material 440 can be configured to have different configurations/characteristics, for example as demonstrated in FIG. 1. It is expressly understood the cannula body 420 can be configured with different particle characteristics at different locations within the cannula body 420. Thus, is it contemplated one side of the cannula body 420 may emit, for example, more light than an other side. Moreover, the cannula body 420 response characteristics within the cannula body 420 or the luminescent material 440 (being distributed within the cannula body 420), may vary according to design preference.

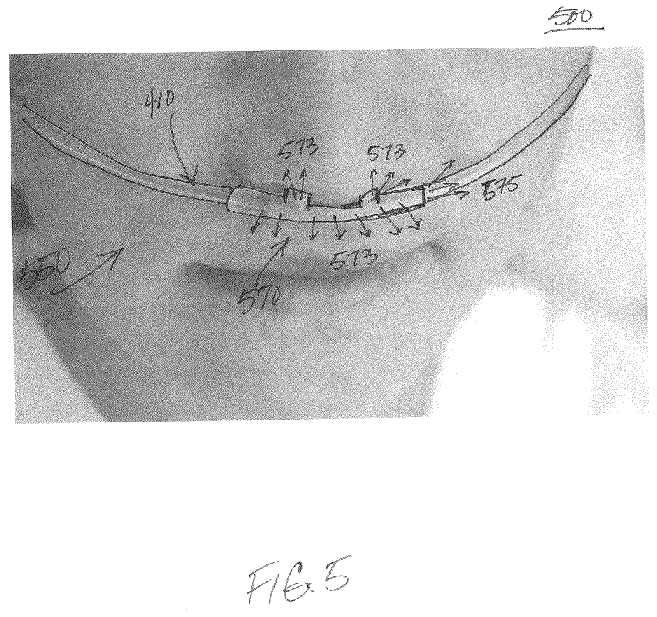

FIG. 5 is an illustration 500 of the exemplary embodiment 410 of FIG. 4 being used by a patient 550. For illustrative purposes only, this example shows an activated state of luminosity 570. Light rays 573 and 575 are illustrated to demonstrate a possible configuration where the luminosity 570 is displayed at different angles or projections as well as a desired color differentiation. As noted above, luminosity 570 may also include non-light characteristics that are representative of the embedded particles. Aspects of this FIG. are self-explanatory in view of the above descriptions.

As described above, the provided FIGS. are in the context of a medical use, however, aspects of the various embodiments may be easily applied to non-medical fields without departing from the spirit and scope of this disclosure.

As non-limiting examples, in certain embodiments, optical responses generated by optically responsive materials associated with an underlying device may be detected and used as inputs to systems, software, or automated mechanisms, including closed-loop systems. For example, in emergency, transport, military, or field environments, optical responses may be used to trigger system behaviors, alerts, or automated responses.

Similarly, such optical responses may be used to influence or affect operation of pumps or medical devices, including peristaltic, infusion, suction, dialysis, or similar systems, based on detected conditions. When scanning or upon detection of coded optical layer, this information can be utilized for confirmation of delivery, transfer, or completion, which may be provided as input to routing, logistics, or workflow processes, including specimen handling, material delivery, or transport systems.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

What is claimed is:

1. An optically responsive medical-grade article, comprising:
a body formed of a polymeric material suitable for use in a medical environment; and
an optically responsive component embedded within the body,
wherein the optically responsive component is configured to produce a machine-detectable optical response in response to an operational condition of the article, and
wherein the optical response is configured to be detected by a sensing system and used to associate information indicative of the operational condition, without reliance on human visual observation.

2. The article of claim 1, wherein the optically responsive component comprises luminescent particles dispersed within the body.

3. The article of claim 2, wherein the luminescent particles comprise phosphorescent, fluorescent, electroluminescent, or triboluminescent materials.

4. The article of claim 1, wherein the optically responsive component is embedded to inhibit leaching or migration during use.

5. The article of claim 1, wherein the machine-detectable optical response varies in response to at least one operational condition selected from fluid flow, pressure, temperature, movement, or chemical exposure.

6. The article of claim 1, wherein the optically responsive component is arranged in a defined region configured to encode information detectable by the sensing system.

7. The article of claim 1, wherein the optically responsive component further exhibits an electrostatic or capacitive characteristic detectable by the sensing system.

8. The article of claim 1, wherein the optically responsive component further exhibits a magnetic characteristic detectable independently of visible light emission.

9. The article of claim 1, wherein the article comprises medical tubing, a catheter, a cannula, or a conduit.

10. The article of claim 9, wherein the conduit defines a lumen configured to convey a fluid.

11. A system, comprising:
the optically responsive medical-grade article of claim 1; and
a sensing system configured to detect the machine-detectable optical response produced by the article and to associate the detected optical response with information indicative of the operational condition of the article.

12. The system of claim 11, wherein the sensing system comprises an optical sensor, a camera, or a machine-vision device.

13. The system of claim 11, further comprising a processor configured to process the associated information.

14. The system of claim 13, wherein the processor generates a signal based on the associated information.

15. A method of monitoring an operational condition of a medical-grade article, comprising:
providing the optically responsive medical-grade article of claim 1;
subjecting the article to an operational condition;
detecting, using a sensing system, a machine-detectable optical response produced by the article; and
associating the detected optical response with information indicative of the operational condition.

16. The method of claim 15, wherein detecting is performed without reliance on human visual observation.

17. The method of claim 15, further comprising processing the associated information to generate a signal.

18. The method of claim 17, further comprising using the signal to verify or influence operation of a medical device or process associated with the article.

19. The system of claim 11, wherein detection occurs continuously, periodically, intermittently, or in response to a triggering condition.

20. The method of claim 15, wherein the associated information is recorded or stored as part of a verification or documentation process.
